(19) United States
(12) Patent Application Publication
(10) Pub. No.: US 2015/0335254 A1
(43) Pub. Date: Nov. 26, 2015

(54) Application For Monitoring A Property Of A Surface

(71) Applicants: Steven FASTERT, (US); Gregory LEVESQUE, (US); Nicholas MCMAHON, (US); Conor RAFFERTY, (US)

(72) Inventors: Steven Fastert, Chelmsford, MA (US); Gregory Levesque, Cambridge, MA (US); Nicholas McMahon, Cambridge, MA (US); Conor Rafferty, Newton, MA (US)

(73) Assignee: MC10, Inc., Lexington, MA (US)

(21) Appl. No.: 14/758,946

(22) PCT Filed: Aug. 1, 2014
(86) PCT No.: PCT/US2014/010740

§ 371 Date: Jul. 1, 2015

Related U.S. Application Data
(60) Provisional application No. 61/750,269, filed on Jan. 8, 2013.
(60) Provisional application No. 61/750,587, filed on Jan. 9, 2013.
(60) Provisional application No. 61/750,596, filed on Jan. 9, 2013.

(51) Int. Cl. A61B 5/01; G01J 5/00; G01N 21/33; A61B 5/00; G01K 13/00

(52) U.S. Cl. CPC A61B 5/01; A61B 5/0008; A61B 5/7275; A61B 5/445; G01K 13/002; G01N 21/33; G01J 5/00

(57) ABSTRACT

The systems, methods apparatus and devices are provided for monitoring a property of an object or an individual using a conformal sensor device mounted to a portion of a surface of the object or the individual. The method includes receiving data indicative of at least one measurement of at least one sensor component of a conformal sensor device that substantially conforms to contours of the surface to provide a degree of conformal contact. The method includes analyzing the data to generate at least one parameter indicative of the property of the surface and the degree of the conformal contact. The data indicative of the at least one measurement includes data indicative of the degree of the conformal contact. The property of the surface is at least one of: an amount of exposure of the surface to the electromagnetic radiation, and a temperature of the object or the individual.

### CROSS-REFERENCE TO RELATED PATENT APPLICATIONS

This application claims priority U.S. provisional application No. 61/750,269, filed Jan. 8, 2013, entitled “UV SENSOR & TEMPERATURE SENSOR DEVICES AND PATCHES,” U.S. provisional application No. 61/750,587, filed Jan. 9, 2013, entitled “TEMPERATURE SENSOR APP,” and U.S. provisional application No. 61/750,596, filed Jan. 9, 2013, entitled “TEMPERATURE SENSOR APP,” each of which is hereby incorporated herein by reference in its entirety.

### BACKGROUND OF THE DISCLOSURE

Effort is being made to develop electronics for application in monitoring properties of a surface, including in the field of skin care and skin health. For example, skin cancer is the most commonly diagnosed type of cancer and the majority of skin cancer can be linked to over-exposure to ultraviolet (UV) rays from the sun or sun-beds. Increased awareness may assist in the prevention of overexposure to UV electromagnetic rays, reducing the risk of skin cancer.

Temperature measurements can be useful for monitoring an individual's health. For example, an elevated temperature can be indicative of a fever condition or overexertion. In other examples, depressed temperatures can be indicative of hypothermia.

The use of electronics in some medical-related applications can be hampered by the boxy, rigid way that much electronics are designed and packaged. Biological tissue is mainly soft, pliable and curved. By contrast, boxy, rigid electronics can be hard and angular, which could affect the measurement of tissue.

Such rigid electronics also may limit applications in non-medical-based systems.

### SUMMARY OF THE DISCLOSURE

In view of the foregoing, systems and methods are provided for monitoring the properties of an object or individual. The systems and method disclosed herein can be used to measure values indicative of, e.g., temperature or exposure to electromagnetic radiation. In some implementations, the system can be disposed into conformal electronics that can be coupled directly to an object or individual, such as being disposed on clothing and protective gear. The system provides an application on a computing device for analyzing data from sensor measurements.

The example systems, methods apparatus and devices herein provide for monitoring a property of an object or an individual using a conformal sensor device mounted to a portion of a surface of the object or the individual. The method includes receiving data indicative of at least one measurement of at least one sensor component of a conformal sensor device that substantially conforms to contours of the surface to provide a degree of conformal contact. The method includes analyzing the data to generate at least one parameter indicative of the property of the surface and the degree of the conformal contact. The data indicative of the at least one measurement includes data indicative of the degree of the conformal contact. The property of the surface is at least one of: an amount of exposure of the surface to the electromagnetic radiation, and a temperature of the object or the individual.

According to the principles herein, a system is provided to monitor a property of an object or an individual using a conformal sensor device mounted to a portion of a surface of the object or the individual. In the example system includes at least one memory for storing processor executable instructions, and a processing unit for accessing the at least one memory and executing the processor executable instructions. The processor executable instructions includes a communication module to receive data indicative of at least one measurement of at least one sensor component of the conformal sensor device, and an application comprising an analysis engine to analyze the data to generate at least one parameter indicative of the property of the surface and the degree of the conformal contact. The conformal sensor device includes the at least one sensor component to obtain the at least one measurement of at least one of: (a) an amount of electromagnetic radiation incident on the at least one sensor component, the electromagnetic radiation having frequencies in the infrared, visible or ultraviolet regions of the electromagnetic spectrum, and (b) a temperature of a portion of the surface. The conformal sensor device substantially conforms to contours of the surface to provide a degree of conformal contact. The data indicative of the at least one measurement includes data indicative of the degree of the conformal contact. The property of the surface is at least one of: an amount of exposure of the surface to the electromagnetic radiation, and a temperature of the object or the individual.

In an example, the application further includes a display module to display the data and/or the at least one parameter.

In an example, the conformal sensor device further includes at least one communication interface to transmit the data indicative of the at least one measurement.

In another example, the conformal sensor device further includes a flexible and/or stretchable substrate, and the at least one sensor component is disposed on the flexible and/or stretchable substrate.

In an example, the surface is a portion of a tissue, a fabric, a plant, an artwork, paper, wood, a mechanical tool, or a piece of equipment.

In an example, the conformal sensor device further includes at least one stretchable interconnect to electrically couple the at least one sensor component to at least one other component of the conformal sensor device. The at least one other component can be at least one of: a battery, a transmitter, a transceiver, an amplifier, a processing unit, a charger regulator for a battery, a radio-frequency component, a memory, and an analog sensing block.

In an example, the communication module includes a near-field communication (NFC)-enabled component to receive the data.

In an example, the communication module implements a communication protocol based on Bluetooth® technology, Wi-Fi, Wi-Max, IEEE 802.11 technology, a radio frequency (RF) communication, an infrared data association (IrDA) compatible protocol, or a shared wireless access protocol (SWAP).

In an example, the analysis engine analyzes the data by comparing the data to a calibration standard.

In an example, the data can include data indicative of the amount of electromagnetic radiation incident on the at least one sensor component, and the comparing provides the indication of the amount of exposure of the surface to the electromagnetic radiation. The calibration standard can include a correlation between values of the data and known amounts of exposure of surfaces to the electromagnetic radiation.

In an example, the data can include data indicative of the temperature of the portion of the surface, and the comparing provides the indication of the temperature of the object or the individual. The calibration standard can include a correlation between values of the data and computed temperatures of objects or individuals.

In an example, the system can further include at least one memory to store the data and/or the at least one parameter.

According to the principles herein, a method is provided to monitor a property of an object or an individual using a conformal sensor device mounted to a portion of a surface of the object or the individual. The method includes receiving, using a communication interface, data indicative of at least one measurement of at least one sensor component of the conformal sensor device, the conformal sensor device, and analyzing the data, using a processing unit executing an application, to generate at least one parameter indicative of the property of the surface and the degree of the conformal contact. The conformal sensor device includes the at least one sensor component to obtain the at least one measurement of at least one of: (a) an amount of electromagnetic radiation incident on the at least one sensor component, the electromagnetic radiation having frequencies in the infrared, visible or ultraviolet regions of the electromagnetic spectrum, and (b) a temperature of a portion of the surface. The conformal sensor device substantially conforms to contours of the surface to provide a degree of conformal contact. The data indicative of the at least one measurement includes data indicative of the degree of the conformal contact. The property of the surface is at least one of: an amount of exposure of the surface to the electromagnetic radiation, and a temperature of the object or the individual.

In an example, the method further includes storing to at least one memory the data and/or the at least one parameter. The method can further include displaying, using a display of the application, the data and/or the at least one parameter.

In an example, the analyzing the data includes comparing the data to a calibration standard.

In an example, the data includes data indicative of the amount of electromagnetic radiation incident on the at least one sensor component, and the comparing provides the indication of the amount of exposure of the surface to the electromagnetic radiation. The calibration standard can include a correlation between values of the data and known amounts of exposure of surfaces to the electromagnetic radiation.

In an example, the data includes data indicative of the temperature of the portion of the surface, and the comparing provides the indication of the temperature of the object or the individual. The calibration standard can include a correlation between values of the data and computed temperatures of objects or individuals.

According to the principles herein, at least one non-transitory computer-readable medium is provided having code representing processor-executable instructions encoded thereon, the processor-executable instructions including instructions that, when executed by one or more processing units, perform a method for monitoring a property of an object or an individual using a conformal sensor device mounted to a portion of a surface of the object or the individual. The method includes receiving, using a communication interface, data indicative of at least one measurement of at least one sensor component of the conformal sensor device, the conformal sensor device, and analyzing the data, using a processing unit executing an application, to generate at least one parameter indicative of the property of the surface and the degree of the conformal contact. The conformal sensor device includes the at least one sensor component to obtain the at least one measurement of at least one of: (a) an amount of electromagnetic radiation incident on the at least one sensor component, the electromagnetic radiation having frequencies in the infrared, visible or ultraviolet regions of the electromagnetic spectrum, and (b) a temperature of a portion of the surface. The conformal sensor device substantially conforms to contours of the surface to provide a degree of conformal contact. The data indicative of the at least one measurement includes data indicative of the degree of the conformal contact. The property of the surface is at least one of: an amount of exposure of the surface to the electromagnetic radiation, and a temperature of the object or the individual.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 shows a block diagram of an example system, according to the principles herein.

FIG. 2 shows a block diagram of an example conformal sensor device, according to the principles herein.

FIG. 3 shows examples of properties of an individual that may be monitored, according to the principles herein.

FIG. 4 shows an example patch, according to the principles herein.

FIG. 5 shows a block diagram of an example computing device, according to the principles herein.

FIG. 6A shows the architecture of an example computer system, according to the principles herein.

FIG. 6B shows a flowchart of an example method, according to the principles herein.

FIG. 7 shows an example EM App, according to the principles herein.

FIG. 8 shows an example graphic display of an example EM App, according to the principles herein.

FIG. 9 shows an example table that the user can navigate to using the example EM App, according to the principles herein.

FIG. 10 shows an example graphic display of data that is collected from an example conformal sensor device, according to the principles herein.

FIG. 11 shows an example display of the example EM App, according to the principles herein.

FIG. 12 shows an example settings page of the example EM App, according to the principles herein.

FIG. 13 shows an example patch information display of the example EM App, according to the principles herein.

FIG. 14 shows an example display of the example EM App, according to the principles herein.

FIG. 15 shows an example temperature App, according to the principles herein.

FIG. 16 shows example display of the example temperature App, according to the principles herein.

FIG. 17 shows an example table that the user can navigate to using the example temperature App, according to the principles herein.

FIG. 18 shows an example graphical plot of the example temperature App, according to the principles herein.

FIG. 19 shows an example settings page of the example temperature App, according to the principles herein.

FIG. 20 shows an example patch information display of the example temperature App, according to the principles herein.

FIG. 21 shows an example alarm display of the example temperature App, according to the principles herein.

FIG. 22 shows an example of a settings page of the example temperature App, according to the principles herein.

### DETAILED DESCRIPTION

It should be appreciated that all combinations of the concepts described in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the particular concepts disclosed herein.

Following below are more detailed descriptions of various concepts related to, and embodiments of, inventive methods, apparatus and systems for monitoring a property of an object or an individual using a conformal sensor device mounted to a portion of a surface of the object or the individual. It should be appreciated that various concepts introduced above and described in greater detail below may be implemented in any of numerous ways, as the disclosed concepts are not limited to any particular manner of implementation. Examples of specific implementations and applications are provided primarily for illustrative purposes.

As used herein, the term “includes” means includes but is not limited to, the term “including” means including but not limited to. The term “based on” means based at least in part on.

The disclosure relates to systems, methods and apparatus that are used for monitoring a property of an object or an individual using a conformal sensor device mounted to a portion of a surface of the object or the individual. The conformal sensor device includes at least one sensor component for performing the measurements. The measurements can be of the temperature of a portion of the surface, and/or an amount of electromagnetic radiation incident on the sensor component. In an example, the electromagnetic radiation is of frequencies in the infrared, visible or ultraviolet regions of the electromagnetic spectrum. The conformal sensor device substantially conforms to contours of the surface to provide a degree of conformal contact. The measurements of the at least one sensor component provides data that can be analyzed to provide at least one parameter indicative of the property of the surface. Non-limiting examples of the property of the object or individual that can be determined based on the analysis include an indication of the amount of exposure of the surface to the electromagnetic radiation, and the temperature of the object or the individual. Analysis of the data also can provide information indicative of the degree of conformal contact of the conformal sensor device with the contours of the surface.

For any of the example systems, methods, apparatus and devices described herein, the object on which the conformal sensor device is mounted can be a human subject and/or a body part of the human subject. For example, in some implementations the object can be a subject's head, arm, foot, chest, abdomen, and/or shoulder. In some examples, the object can be an inanimate object.

An example system according to the principles herein provides for monitoring a property of an object or an individual using a conformal sensor device mounted to a portion of a surface of the object or the individual. The example system employs an application running on a mobile communication device. Non-limiting examples of such mobile communication devices include a smartphone, such as but not limited to an iPhone®, a BlackBerry®, or an Android-based smartphone, a tablet, a slate, an electronic-reader (e-reader), a digital assistant, or other electronic reader or hand-held, portable, or wearable computing device, or any other equivalent device, an Xbox®, a Wii®, or other game system(s). The conformal sensor device is communicatively coupled to the mobile communication device. The conformal sensor device includes at least one sensor component to takes measurements, such as but not limited to measurements of the temperature of a portion of the surface, or the amount of electromagnetic radiation incident on the sensor component. The mobile communication device receives the data indicative of the measurement(s). The mobile communication device includes an application that analyzes the data to determine at least one parameter indicative of the property of the surface, such as but not limited to an indication of the amount of exposure of the surface to the electromagnetic radiation, and the temperature of the object or the individual.

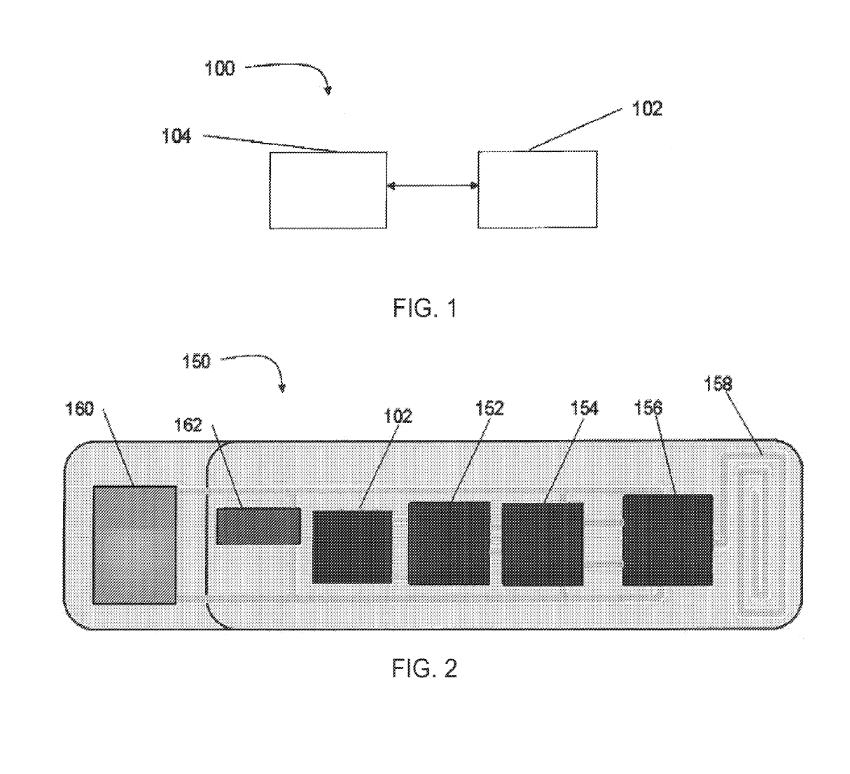

FIG. 1 shows a block diagram of a non-limiting example system according to the principles herein. The example system 100 includes at least one conformal sensor device 102 that includes at least one sensor component to provide a measurement as described herein. For example, the measurement can be of the temperature of a portion of a surface or of an amount of electromagnetic radiation that the at least one sensor component is exposed to (including electromagnetic radiation in the visible spectrum or ultra-violet light). The conformal sensor device 102 can include at least one other component. In an example implementation, the at least one other component can be a processing unit. In an example implementation, the at least one component can be configured to supply power to the conformal sensor device 102. For example, the at least one other component can include a battery or any other energy storage device that can be used to supply a potential.

As shown in FIG. 1, the conformal sensor device 102 is communicatively coupled to an external computing device 104. Non-limiting examples of the computing device 104 include a smartphone, a tablet, a slate, an e-reader, a digital assistant, or any other equivalent device, including any of the mobile communication devices described hereinabove. As an example, the computing device 104 can include a processor unit that is configured to execute an application that includes an analysis module for analyzing the data signal from the conformal sensor device.

In an example implementation, the conformal sensor device 102 includes at least one other component that is configured to transmit a signal from the apparatus to an example computing device 104. For example, the at least one component can include a transmitter or a transceiver configured to transmit a signal including data indicative of a measurement by the at least one sensor component to the example computing device 104.

In an example, the conformal sensor device 102 can include at least one sensor component to measure an electrical property of the surface. For example, a capacitive-based measurement of the electrical properties of tissue can be used to provide a measure of the state of hydration of the tissue. In an example implementation, the at least one other component can include at least one processor unit.

In an example, the conformal sensor device includes the at least one sensor disposed on a flexible and/or stretchable substrate. In some examples, the conformal sensor device is encapsulated in a flexible and/or stretchable encapsulant material. According to the principles herein, the substrate and/or encapsulant can include one more of a variety of polymers or polymeric composites, including polyimides, polyesters, a silicone or siloxane (e.g., polydimethylsiloxane (PDMS)), a photo-patternable silicone, a SU8 or other epoxy-based polymer, a polydioxanone (PDS), a polystyrene, a parylene, a parylene-N, an ultrahigh molecular weight polyethylene, a polyether ketone, a polyurethane, a polyactic acid, a polyglycolic acid, a polytetrafluoroethylene, a polyamic acid, a polymethyl acrylate, or any other flexible or stretchable materials, including compressible aerogel-like materials, and amorphous semiconductor or dielectric materials. In some examples described herein, the conformal sensor device can include non-flexible electronics disposed on the substrate or disposed between flexible or stretchable layers. In another non-limited example, the substrate and/or encapsulant can be formed from a silicone such as but not limited to SORTACLEAR® silicone, SOLARIS® silicone, or ECOFLEX® silicone (all available from Smooth-On, Inc., Easton, Pa.). In an example, the encapsulation layer has a Young's modulus of about 100 MPa or less. In an example implementation where an example conformal sensor device is configured to detect electromagnetic radiation in the IR or visible regions of the electromagnetic spectrum, an encapsulation layer formed from a polyimide may be used, since a polyimide can be configured to absorb ultraviolet electromagnetic frequencies. In an example, an encapsulation layer formed from a polyimide may be used for an example conformal sensor device configured to detect electromagnetic radiation in the UV region of the electromagnetic spectrum.

In an example, the electronics of the conformal sensor device can include at least one stretchable interconnect to electrically couple the at least one sensor component to at least one other component of the conformal sensor device. In some examples, the at least one other component is at least one of: a battery, a transmitter, a transceiver, an amplifier, a processing unit, a charger regulator for a battery, a radio-frequency component, a memory, and an analog sensing block.

In an example, the conformal sensor device can include at least one sensor component, such as but not limited to a temperature sensor or an electromagnetic radiation sensor. The at least one sensor component can include an accelerometer and/or a gyroscope. In such examples, the accelerometer and/or gyroscope can be commercially available, including “commercial off-the-shelf” or “COTS.” The accelerometers may include piezoelectric or capacitive components to convert mechanical motion into an electrical signal. A piezoelectric accelerometer may exploit properties of piezoceramic materials or single crystals for converting mechanical motion into an electrical signal. Capacitive accelerometers can employ a silicon micro-machined sensing element, such as a micro-electrical-mechanical system, or MEMS, sensing element. A gyroscope can facilitate the determination of refined location and magnitude detection. As a non-limiting example, a gyroscope can be used for determining the tilt or inclination of the object to which it is coupled. As another example, the gyroscope can be used to provide a measure of the rotational velocity or rotational acceleration of the object. For example, the tilt or inclination can be computed based on integrating the output (i.e., measurement) of the gyroscope.

FIG. 2 shows a block diagram of a non-limiting example conformal sensor device 150 according another implementation of the principles herein. The example system 150 includes at least one sensor component 102 that can be used to perform a measurement. The measurement can be of an amount of exposure of a surface to electromagnetic radiation, of a temperature of a portion of the surface, or of the electrical properties of the surface through a capacitive-based measurement. In the non-limiting example of FIG. 2, the at least one other component includes an analog sensing block 152 that is coupled to the at least one sensor component 102 and at least one processor unit 154 that is coupled to the analog sensing block 152. The at least one other component includes a memory 156. For example, the memory 156 can be a non-volatile memory. As a non-limiting example, the memory 156 can be mounted as a portion of a RF chip. The at least one other component also includes a transmitter or transceiver 158. The transmitter or transceiver 158 can be used to transmit data from the at least one sensor component 102 to the example computing device 104 (not shown). The example system 150 of FIG. 2 also includes a battery 160 and a charge regulator 162 coupled to battery 160. The charge regulator 162 and battery 160 are coupled to the processor unit 154 and memory 156.

A non-limiting example use of system 150 is as follows. Battery 160 provides power for the apparatus 102 to perform the measurements. The processor unit 154 activates periodically, stimulates the analog sensing block 152, which conditions the signal and delivers it to an A/D port on the processor unit 154. The data from apparatus 102 is stored in memory 156. In an example, when a near-field communication (NFC)-enabled computing device 104 (not shown) is brought into proximity with the system 150, data is transferred to the handheld device, where it is interpreted by application software of the handheld device. The data logging and data transfer can be asynchronous. For example, data logging can occur each minute while data transfer may occur episodically.

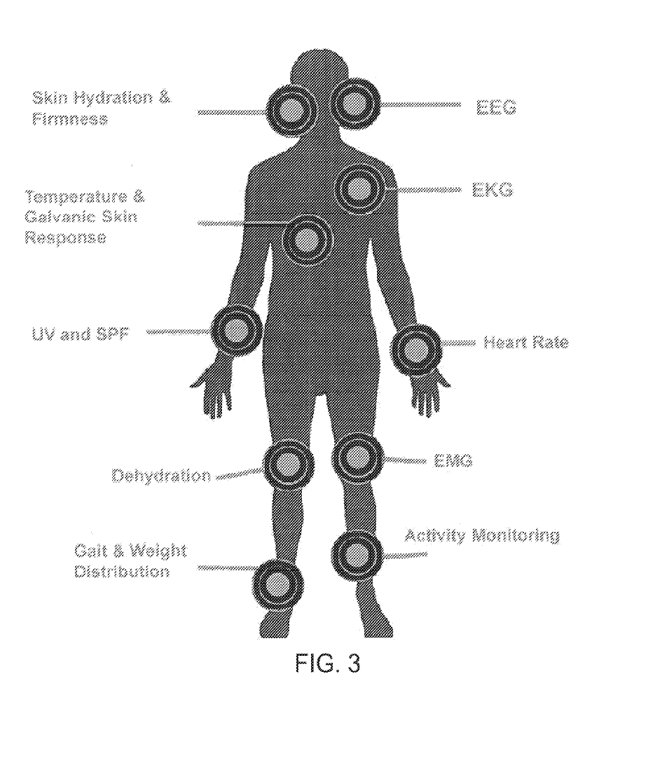

An example conformal sensor device according to the principles described herein can be used to monitor properties in conjunction with a wide range of other on-body sensors. Non-limiting examples of properties that may be monitored using one or more of the conformal sensor devices described herein are shown in FIG. 3. For example, an example conformal sensor device herein can include at least one sensor component according to the principles herein for measuring an amount of IR, visible or UV light exposure of the tissue, or an amount of sun protection factor (SPF) provided by a product applied to the tissue. As yet another example, an apparatus herein can be configured to include at least one hydration sensor for measuring a hydration level of the tissue. As another example, an apparatus herein can be configured to include at least one temperature sensor for measuring the temperature of the tissue.

The apparatus and systems of the technology platform described herein support conformal electronics that can be used to log sensor data at very low power levels over extended periods, while providing wireless communication with external computing devices (including handheld devices). The conformal electronics include on-body electronics and electronics that conform to other surfaces, including paper, wood, leather, fabric (including artwork or other works on canvas), a plant or a tool.

The technology platform described herein supports conformal electronics that can be used to monitor an amount of electromagnetic radiation that a surface is exposed to. In an example, the sensor components are UV sensors that allow the continuous recording of UVA and UVB exposure. In a non-limiting example, an example conformal sensor device described herein can be configured as a IR/visible/UV sensor that records the amount of electromagnetic radiation that a surface is exposed to, and transmits the data measurement to the example computing device.

In an example, any sensor device described in U.S. patent application Ser. No. 13/603,290, filed Sep. 4, 2012, entitled “ELECTRONICS FOR DETECTION OF A CONDITION OF TISSUE” or U.S. patent application Ser. No. 13/631,739, filed Sep. 28, 2012, entitled “ELECTRONICS FOR DETECTION OF A PROPERTY OF A SURFACE,” each of which is incorporated herein by reference in its entirety including drawings, can be implemented as a conformal sensor device according to the principles of any of the examples described herein.

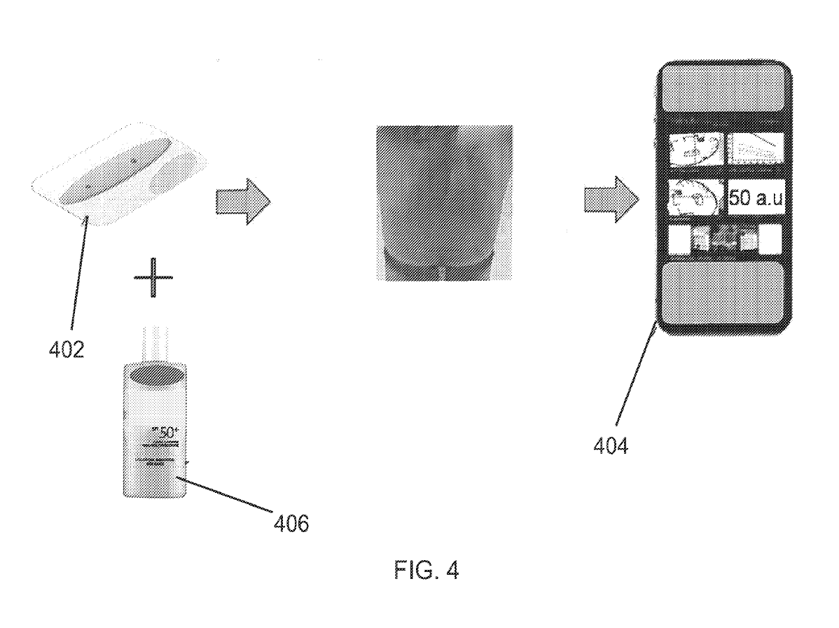

In a non-limiting example, a conformal sensor device according to any of the principles described herein can be mounted to the surface as a part of a patch. The surface can be a part of a surface of paper, bottles or other packaging, wood, leather, fabric, including artwork or other works on canvas, a plant or a tool. An example of a patch 402 that can include at least one of any of the apparatus described herein is shown in FIG. 4. The patch 402 may be applied to the surface, such as but not limited to a portion of skin. An example computing device 404 can be used to receive the data in connection with the electrical measurement performed by the example conformal sensor device of the patch 402. For example, the patch 402 can include a transmitter or transceiver to transmit a signal to the example computing device 404.

In any example herein, the transmission of the data from the conformal sensor device to the computing device may be dependent on their proximity to each other. For example, the computing device may be configured to receive the data when the computing device is within a few centimeters of the conformal sensor device. A user may facilitate the transfer of data from the conformal sensor device (including one disposed on a patch) by positioning the computing device in proximity to the conformal sensor device.

As described in greater detail below, the computing device can include an application (an “App”) to perform such functionalities as analyzing the data. For example, the data from the at least one sensor component can be analyzed as described herein by a processor executing the App on the example computing device 404 to provide the indication of the property of the object or individual. For example, the analysis of the data can provide at least one parameter indicative of a property such as but not limited to an exposure of the surface to electromagnetic radiation, the SPF factor of a product applied to the surface, the UV Index (UVI) applied to the surface, the change in electromagnetic (EM) radiation applied to the surface due to atmospheric conditions versus an external measurement of the same EM radiation, or a condition of the surface, a temperature of the object or individual, a hydration state of the surface, according to the principles described herein.

In some examples, the analysis of the data can provide at least one parameter indicative of a property such as but not limited to the UV Index (UVI) applied to the surface, or the change in electromagnetic (EM) radiation applied to the surface due to atmospheric conditions versus an external measurement of the same EM radiation. In an example, the analysis engine of the App can be implemented to compare local EM measurements to remote EM predictions, projections or measurements (such as but not limited to those provided by a centralized weather service). In another example, the analysis engine of the App can be implemented to compare the UVI from the centralized weather service (such as but not limited to the Weather Channel) for a given geographical area to the actual UVI of an individual living in the given geographical area. In another example, the analysis engine of the App can be implemented to compute any differences in the UV exposure of an individual under changing ozone and/or smog conditions.

In some examples, the App can be implemented to log and/or to track the at least one parameter over time. For example, the App can be implemented to log and/or to track the SPF state of a surface based on episodic sensor measurements over time. That is, the App on the computing device can include processor-executable instructions such that a processor unit of the computing device implements an analysis engine to analyze data indicative of a temperature measurement, an electromagnetic radiation measurement, an electrical measurement, or other sensor component measurement from the conformal sensor device of the patch 402 and provide at least one parameter indicative of a property of the object or individual.

As shown in FIG. 4, the example patch 402 may be used in connection with a substance 406 that is applied to the surface. The substance 406 may be configured to change the condition of the surface, including treating a disease of the surface. For example, the substance 406 may be configured to be applied to the surface to provide protection against the UV or other harmful EM radiation. In this example, the example patch can be configured to perform electrical measurements to provide an indication of UV and/or SPF sensing on the surface, to prevent sun damage and/or to recommend protective products. In another example, the substance 406 may be configured to be applied to the surface to treat a disease or other malformation of the surface. In other examples, the substance 406 can be a pharmaceutical drug, a biologic, or other substance to treat a condition to cause a reduction in temperature of the object or individual. In this example, the example patch can be configured to perform temperature measurements to monitor the temperature of the object or individual.

Over time, e.g., throughout the day, a NFC-enabled computing device can be placed in proximity to the patch 402 to gather the data from the measurements. For example, analysis of the data can facilitate checking how much sun protection still remains.

In an example, the example patch 402 may be a durable sensor patch or a disposable adhesive patch that is configured for comfort and breathability. After use, such as at the end of the day, a consumer may dispose of the disposable adhesive patch, and retain the sensor patch for reuse at a later time. The sensor patch can be re-charged using a charging pad.

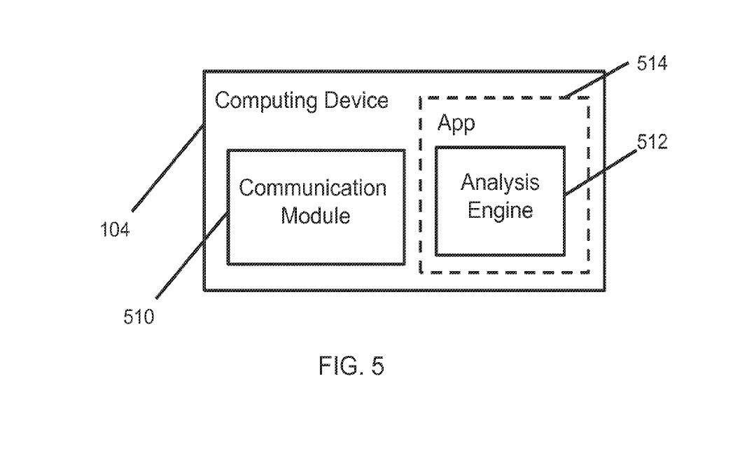

As shown in FIG. 5, the example computing device 104 can include a communication module 510 and an analysis engine 512. The communication module 510 can be implemented to receive data indicative of a measurement of the at least one sensor component of the conformal sensor device. The analysis engine 512 can be implemented to analyze the data to generate at least one parameter indicative of the property of the surface and the degree of the conformal contact. As shown in the example of FIG. 5, the computing device 104 can include processor-executable instructions such that a processor unit can execute an application (an App) 514 that a user can implement to initiate the analysis engine 512. In an example, the processor-executable instructions can include software, firmware, or other instructions.

The example communication module 510 can be configured to implement any wired and/or wireless communication interface by which information may be exchanged between the conformal sensor device 102 and the computing device 104. Non-limiting examples of wired communication interfaces include, but are not limited to, USB ports, RS232 connectors, RJ45 connectors, and Ethernet connectors, and any appropriate circuitry associated therewith. Non-limiting examples of wireless communication interfaces may include, but are not limited to, interfaces implementing Bluetooth® technology, Wi-Fi, Wi-Max, IEEE 802.11 technology, radio frequency (RF) communications, Infrared Data Association (IrDA) compatible protocols, Local Area Networks (LAN), Wide Area Networks (WAN), and Shared Wireless Access Protocol (SWAP).

In any example herein, the App 514 on the computing device 104 can include processor-executable instructions such that the analysis engine analyzes the electrical measurements from the conformal sensor device to provide at least one parameter, such as but not limited to, a temperature of an object or an individual, an amount of exposure of a surface to the electromagnetic radiation, change in exposure to the surface versus an external measurement, a hydration state of a surface, an indication of the status (SPF state) of a surface, a UV Index (UVI) applied to a surface, or a measure of a change in electromagnetic (EM) radiation applied to the surface due to atmospheric conditions versus an external measurement of the same EM radiation. In some example, the App 514 can include processor-executable instructions to provide: (i) product recommendations, (ii) suggestions to re-apply a product, or (iii) present an interface that facilitates the purchase of, or obtaining a sample of, recommended products.

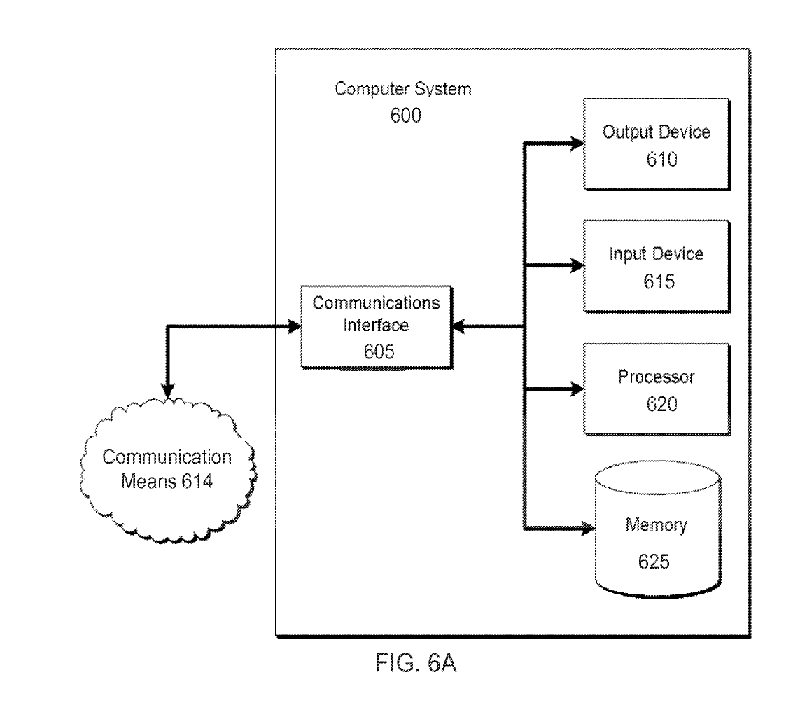

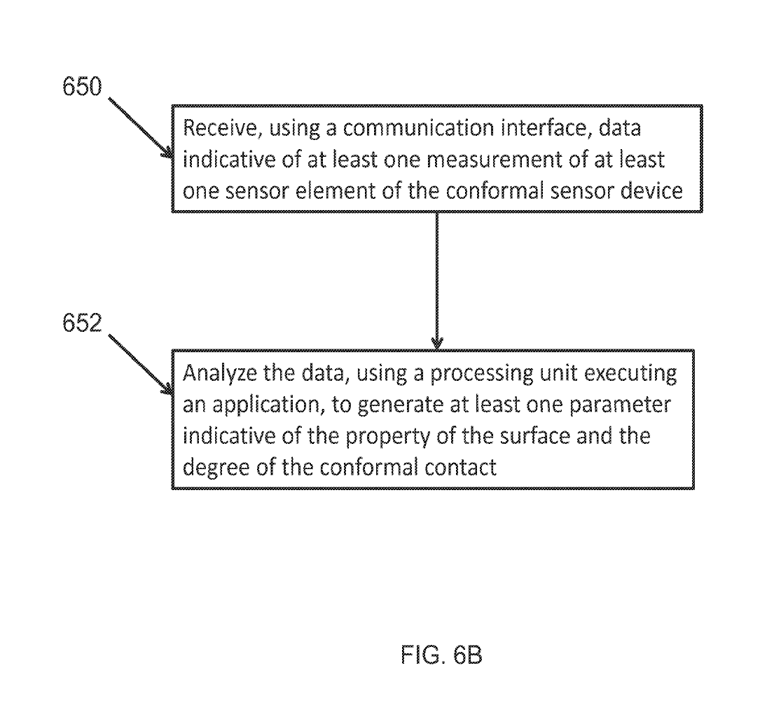

FIG. 6A shows the general architecture of an example computer system 600 that may be employed to implement any of the example systems and methods described herein. The computer system 600 of FIG. 6A includes one or more processors 620 communicatively coupled to at least one memory 625, one or more communications interfaces 605, and one or more output devices 610 (e.g., one or more display units) and one or more input devices 615.

In the computer system 600 of FIG. 6A, the memory 625 may include any computer-readable storage medium, and may store computer instructions such as processor-executable instructions for implementing the various functionalities described herein for respective systems, as well as any data relating thereto, generated thereby, or received via the communications interface(s) or input device(s). The processor(s) 620 shown in FIG. 6A may be used to execute instructions stored in the memory 625 and, in so doing, also may read from or write to the memory various information processed and or generated pursuant to execution of the instructions.

The processor 620 of the computer system 600 shown in FIG. 6A also may be communicatively coupled to or control the communications interface(s) 605 to transmit or receive various information pursuant to execution of instructions. For example, the communications interface(s) 605 may be coupled to a communication means 614, such as but not limited to a wired or wireless network, bus, or other communication means, and may therefore allow the computer system 600 to transmit information to and/or receive information from other devices (e.g., other computer systems). While not shown explicitly in the system of FIG. 6A, one or more communications interfaces facilitate information flow between the components of the system 600. In some example implementations, the communications interface(s) may be configured (e.g., via various hardware components or software components) to provide a website as an access portal to at least some aspects of the computer system 600.

The output devices 610 of the computer system 600 shown in FIG. 6A may be provided, for example, to allow various information to be viewed or otherwise perceived in connection with execution of the instructions. The input device(s) 615 may be provided, for example, to allow a user to make manual adjustments, make selections, enter data or various other information, or interact in any of a variety of manners with the processor during execution of the instructions.

Examples of the systems, methods and operations described herein can be implemented in digital electronic circuitry, or in computer software, firmware, or hardware, including the structures disclosed in this specification and their structural equivalents, or in combinations of one or more thereof. Examples of the systems, methods and operations described herein can be implemented as one or more computer programs, i.e., one or more modules of computer program instructions, encoded on computer storage medium for execution by, or to control the operation of, data processing apparatus. The program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” or “computing device” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, application or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), for example. Devices suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube), plasma, or LCD (liquid crystal display) monitor, for displaying information to the user and a keyboard and a pointing device, e.g., a mouse, touch screen or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

In some examples, a system, method or operation herein can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Example computing system 400 can include clients and servers. A client and server are generally remote from each other and typically interact through a communication network. The relationship of client and server arises by virtue of computer programs running on the respective computers and having a client-server relationship to each other. In some embodiments, a server transmits data to a client device (e.g., for purposes of displaying data to and receiving user input from a user interacting with the client device). Data generated at the client device (e.g., a result of the user interaction) can be received from the client device at the server.

FIG. 6B shows an example method that can be implemented using any of the example systems, apparatus and devices herein. The example method can be used to monitor a property of an object or an individual using a conformal sensor device mounted to a portion of a surface of the object or the individual. The method includes receiving 650, using a communication interface, data indicative of at least one measurement of at least one sensor component of the conformal sensor device. The conformal sensor device includes at least one sensor component to obtain the at least one measurement of at least one of: (a) an amount of electromagnetic radiation incident on the at least one sensor component, the electromagnetic radiation having frequencies in the infrared, visible or ultraviolet regions of the electromagnetic spectrum, and (b) a temperature of a portion of the surface. The conformal sensor device substantially conforms to contours of the surface to provide a degree of conformal contact. The method includes analyzing the data 652, using a processing unit executing an application, to generate at least one parameter indicative of the property of the surface and the degree of the conformal contact. The data indicative of the at least one measurement includes data indicative of the degree of the conformal contact. The property of the surface is at least one of: an amount of exposure of the surface to the electromagnetic radiation, and a temperature of the object or the individual.

### Non-limiting Example Implementations Using Example Apps

Non-limiting example implementations of Apps on computing devices are described. While the Apps are described relative to a series of screenshots and navigation procedures, the subject matter herein is not so limited.

In the non-limiting example implementations described, Apps are described for use with an example conformal sensor device including at least one electromagnetic radiation sensor or at least one temperature sensor. The example Apps are configured as Android® applications for use with a UV light sensing platform or a temperature sensing platform. Although the Apps are developed as Android® Apps, the disclosure is not so limited. The example Apps can be configured to run on other operating systems, including a iOS® operating system or a Windows® operating system.

Non-limiting example components and materials in the example implementations are as follows. The App can be used with a NFC-equipped, internet-connected hand-held computing device (such as but not limited to a Samsung Galaxy Note II®) operating the Android operating system. The App can be configured for download as a sensor App (a *.apk file).

Each different type of computing device running an Android operating system may have a different NFC antenna size and/or location. There a certain amount of time, such as but not limited to about 10 minutes, about 15 minutes, about 20 minutes or more, can be taken to determine the optimal position and/or orientation of the computing device to ensure coupling (synchronization (“sync”)) between the computing device and the patch including the conformal sensor device. An example App can be configured to show an animation requesting a user to “sync the sensor” to the computing device to find the optimal position and/or orientation. Transferring data from the conformal sensor device to the computing device may require a steady connection for a period of time. In any example implementation, the App may be configured to display “Sync Failed” messages to indicate a lack of proper coupling.

In an example implementation, once a successful sync has occurred, the App can be configured to prompt a user, e.g., with a pop-up, to perform at least one of showing the battery status, asking to name the sensor that is synchronized, enter information to specify parameters such as but not limited to a desired sampling frequency, a user's age, or a user's skin type.

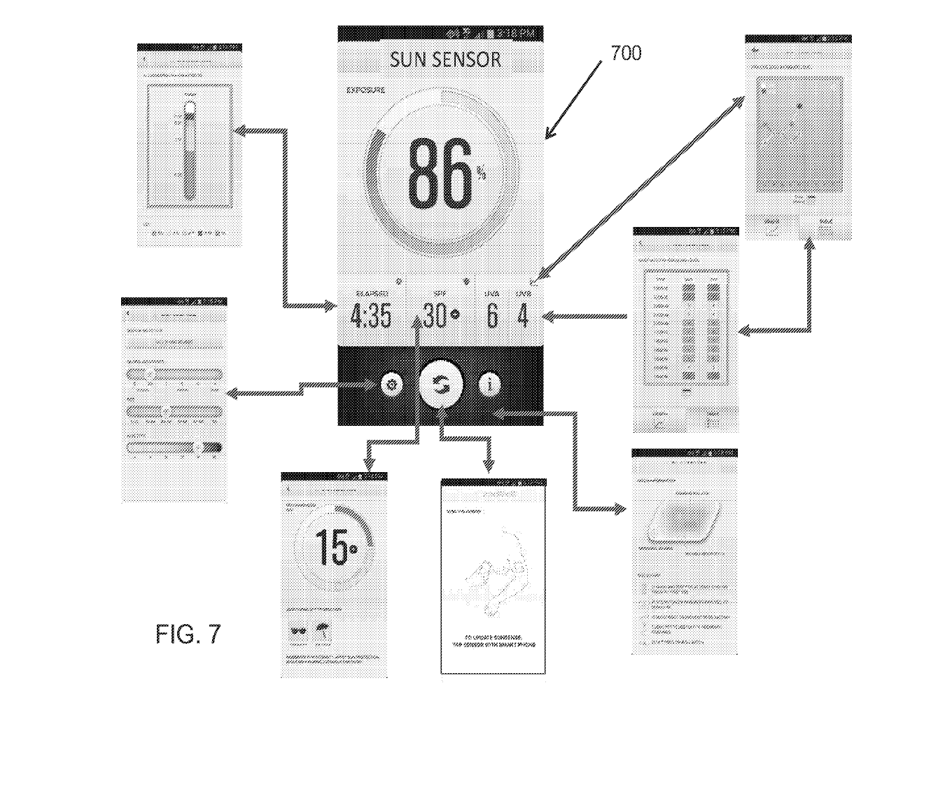

In an example, a computing device with a EM App (see FIG. 7) can be used with the electromagnetic (EM) radiation sensor to interpret UV (sun) exposure for the user. The electromagnetic radiation sensor App can be configured to send instructions to the conformal sensor device to perform the electromagnetic radiation sensor measurements to collect the UV data. In other examples, the App can be configured to use data collected independently and transferred to the computing device using near-field communication (NFC) from a UV Patch including the conformal electromagnetic radiation sensor. In this example, the computations are based on such data as sun intensity (UVA & UVB), time of exposure, and skin type.

Although the user's experience is focused on the EM App running on the computing device, the data and its reliability is focused on the patch including the conformal sensor device, including based on the degree of conformal contact between the patch and the surface of the object or individual. For example, information displayed to the user using a display of the EM App has a similar level of accuracy as the data gathered by the patch, including based on the degree of conformal contact between the patch and the surface. It should be ensured that the patch is charged, operational, and clear of debris that can reduce the degree of conformal contact.

As shown in the example of FIG. 7, the EM App can be developed to be homepage-centric, allowing a user to access more detailed data by clicking on various portions of a homepage. For example, FIG. 7 shows six (6) different buttons (three (3) dynamic buttons & three (3) static buttons) within an example dashboard 700. Using a feature such as a “back” button, positioned either in the upper-left corner of the App or the physical “back” button on the computing device, returns the user to the homepage dashboard 700. The example UV electromagnetic radiation sensor App is depicted as a Sun Sensor App in this example implementation.

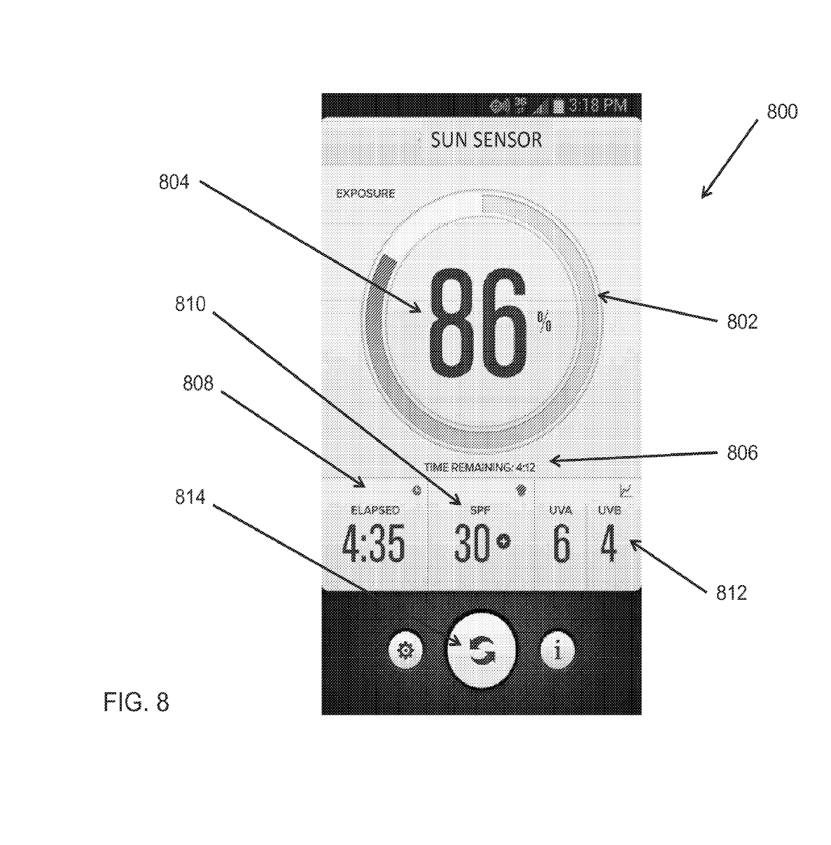

FIG. 8 shows an example graphic that can be displayed as the homepage 800 of the EM App. The homepage 800 shows examples of the types of parameters that can be computed based on the electromagnetic radiation sensor measurements, to indicate properties of the object or individual. For example, the homepage 800 can be configured to display a UV exposure wheel 802 and/or a value of computed exposure percentage 804. These parameters can be computed, using the App's analysis engine, using a UVI-minute dosage specified for each user, e.g., based on s user's skin type. If a user has received 100% exposure as computed using the App's analysis engine, the user may be at risk of a harmful level of UV radiation exposure (with potential for the user experiencing first degree burns).

As also shown in the example homepage 800 can be configured to display results of an a computation of recommended time remaining 806 for safe UV exposure. The time remaining can be computed base don data such as but not limited to a user's cumulative UVI-minute exposure for that day and based on the most recent UVA & UVB levels measured (time of last sync). In an example, when a user has no time remaining base don the projections, the user is considered to have received 100% of their recommended UVI-minute dosage (e.g., as displayed on the exposure wheel 802). Alternatively, when any percentage remains for the exposure wheel, the EM App is configured to cause the time remaining indicator 806 to let a user know the amount of time that the user can be spend outside, based on existing sun conditions. The EM App can be configured to compute a recommended level of UV exposure for a user, e.g., based on a user's indicated skin type (such as based on an industry-wide Fitzpatrick Classification Scale) to define a UVI*minutes dosage for each user.

As also shown in the example homepage 800 can be configured to display at least one of an elapsed time 808 (the time a user has spent in the sun), a value of SPF 810 (a recommended product SPF based on the maximum sun intensity (UVA and UVB) for the day), and values 812 for UVA/UVB (computations of most recent UVI levels for UVA and UVB).

The example EM App can be caused to facilitate data transfer from the conformal sensor device in the patch to the computing device using a “Sync” button 814. For example, the computing device can use NFC to receive data collected since the last synchronization, e.g., transferred from an EEPROM memory of the conformal sensor device. The data may be stored to a data base of the computing device. In other examples, the data can be transferred using other technology such as but not limited to Bluetooth® or Wifi.

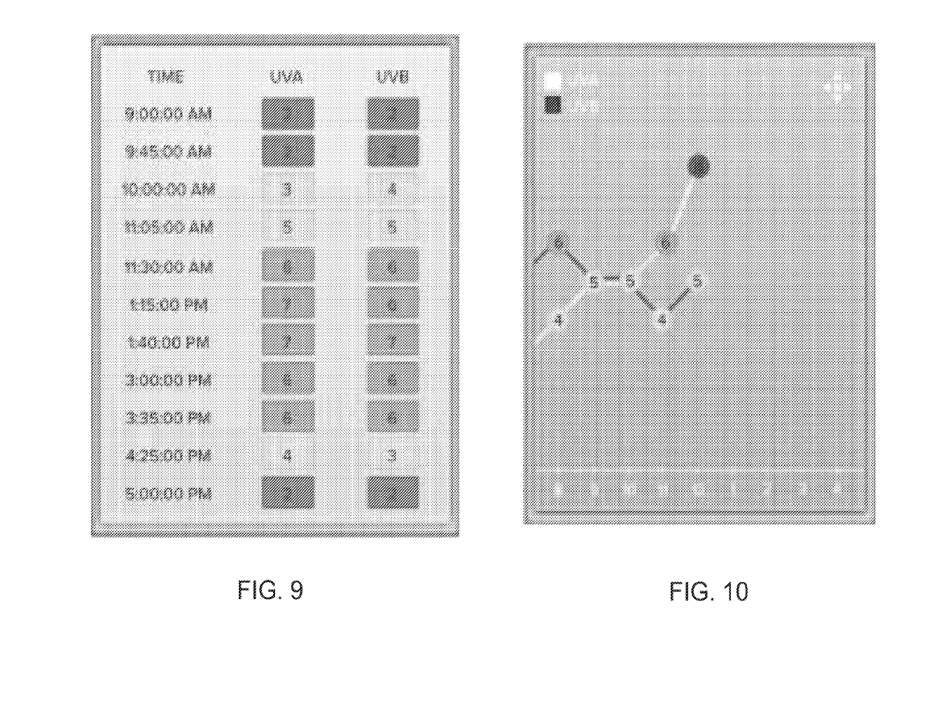

FIG. 9 shows an example table that the user can navigate to using the App, which shows the data that is collected, and the frequency of collection, from the conformal sensor device. FIG. 10 shows an example graphic display of the data that is collected from the conformal sensor device, e.g., to show a set of data collected over a period of time (such as a full day's data).

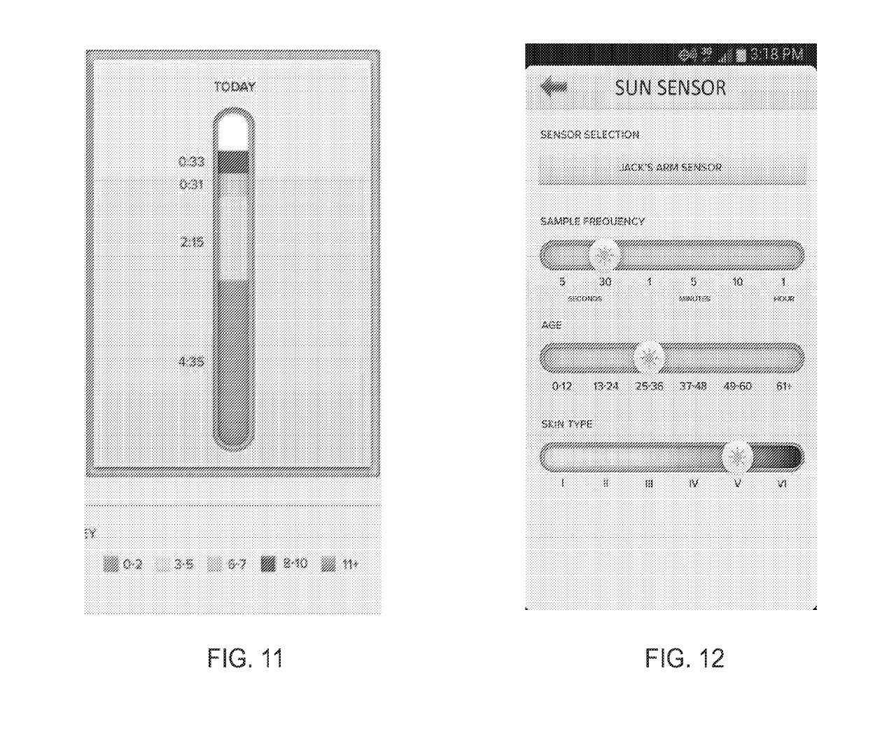

FIG. 11 shows an example display of discrete levels based on UVI level ranges. For example, based on standards set by the World Health Organization's (WHO), a color scheme can be used to indicate UVI levels (green—UVI 0 to 2; yellow—UVI 3 to 5; orange—UVI 6 to 7; red—UVI 8 to 10; purple—UVI 11 or higher). Using the display of the UVI color bar, each region of color can be used to represent s user's exposure to that level of UVI up to the most current time. The bar in the EM App display can be reset at the end of a certain time period (such as but not limited to at the end of each day). The EM App also can be configured to display the bar tagged with the relative time spent in each of the UVI brackets.

FIG. 12 shows an example settings page that the EM App can display to a user. The user is prompted to specify a sample frequency, age, and skin type. Each can be specified using a sliding feature, or by entering numerical values, or other viable display for specifying the values.

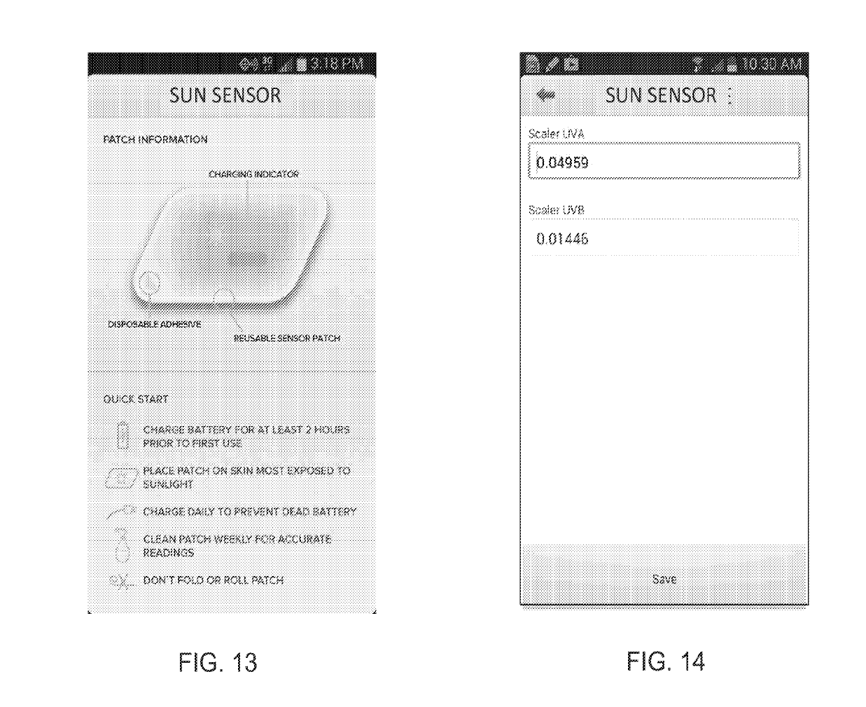

FIG. 13 shows an example patch information display that the user can access on the EM App to provide information about the conformal sensor device and the patch layout. For example, the EM App can be configured to show a display of the different parts of the patch, how they work, and information that a user can use to place and implement the patch on-body.

In a non-limiting example, the analysis engine of the EM App can be configured to compute the UVA, UVB, and UVI levels as follows:

UVA=UVA Scaler*Hex2Dec((([7,0]−Samp Time)<<=8)+[15,8])

*UVA is rounded to the nearest integer. Default UVA Scaler=0.04959

UVB=UVB Scaler*Hex2Dec((([23,16]−Samp Unit)<<=8)+[31,24])

*UVB is rounded to the nearest integer. Default UVB Scaler=0.01446

UVI=25% (UVA)+75% (UVB)

*UVI is rounded to the nearest integer. UVI is never displayed, but is used to calculate cumulative UVI*minutes.

Skin Type Dosages:

| Skin Type | UVI*Minutes |
|---|---|
| I | 62.8 |
| II | 186.92 |
| III | 311.78 |
| IV | 469.16 |
| V | 608.79 |
| VI | 748.41 |

### Elapsed Time:

Elapsed Time=Total time spent in 1UVI or higher

*Elapsed Time resets to 0:00 at the beginning of every day.

Remaining Time:

$$Remaining Time = \frac{Dosage - Cumulated UVI * minutes}{latest UVI level}$$

*Cumulated UVI*minutes resets at the beginning of every day. If the latest UVI level is 0UVI, then it is changed to 1UVI for the purposes of this calculation.

Exposure Percentage:

$$Exposure \% = 100 ( \frac{Cumulated UVI * minutes}{dosage} )$$

*Cumulated UVI*minutes resets at the beginning of every day.

Recommended SPF:

| Max UVI | Rec. SPF |
|---|---|
| 0-2 | 5+ |
| 3-5 | 15+ |
| 6-7 | 30+ |
| 8+ | 45+ |

FIG. 14 shows an example display that can be used to show values used in the computation. For example, FIG. 14 shows an example scaler UVB used in the computation. This value is multiplied to the DECIMAL (base 10) representation of the value on the EEPROM. UVB(UVI)=Scaler*hex2dec(UVB Memory Location). Increasing the scaler UVB value results in the analysis engine computing higher UVB values from the data read.

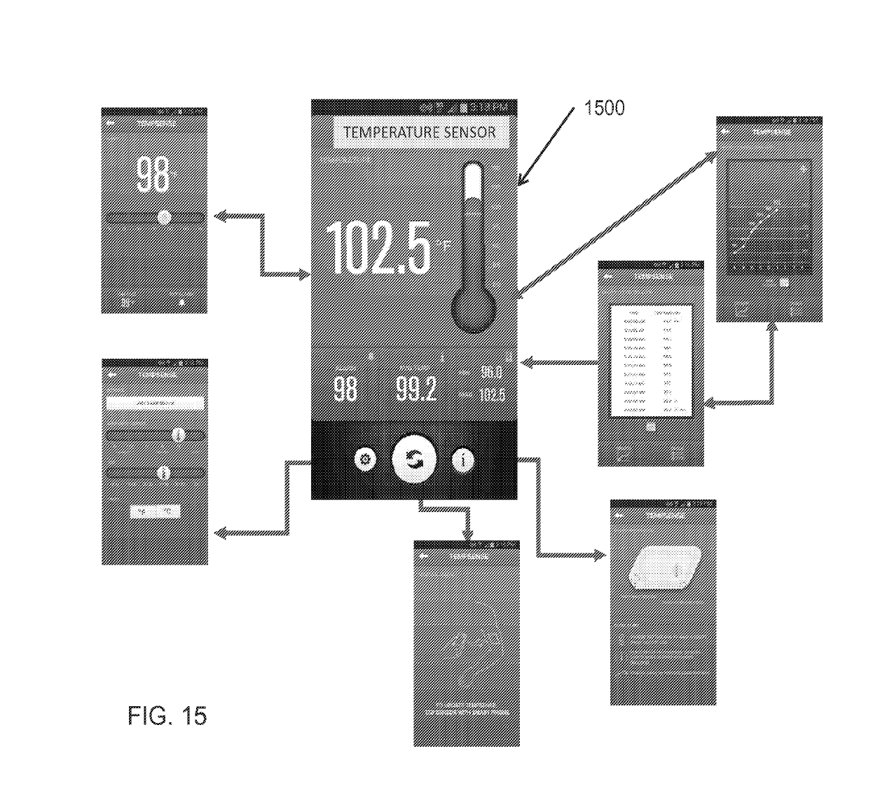

FIG. 15 shows another example implementation of an App, where a computing device with a temperature App is used with a temperature sensor to interpret temperature measurements. The example temperature App is configured based on an Android operating system as described above in connection with the EM App. For example, the temperature App is configured to be based on a homepage 1500 that provides a user with access to data and analysis results by clicking on six (6) different buttons (three (3) dynamic buttons and three (3) static buttons within the dashboard). Pressing the back button (either in the upper-left corner of the App or the physical back button on the device), returns the user to the homepage 1500.

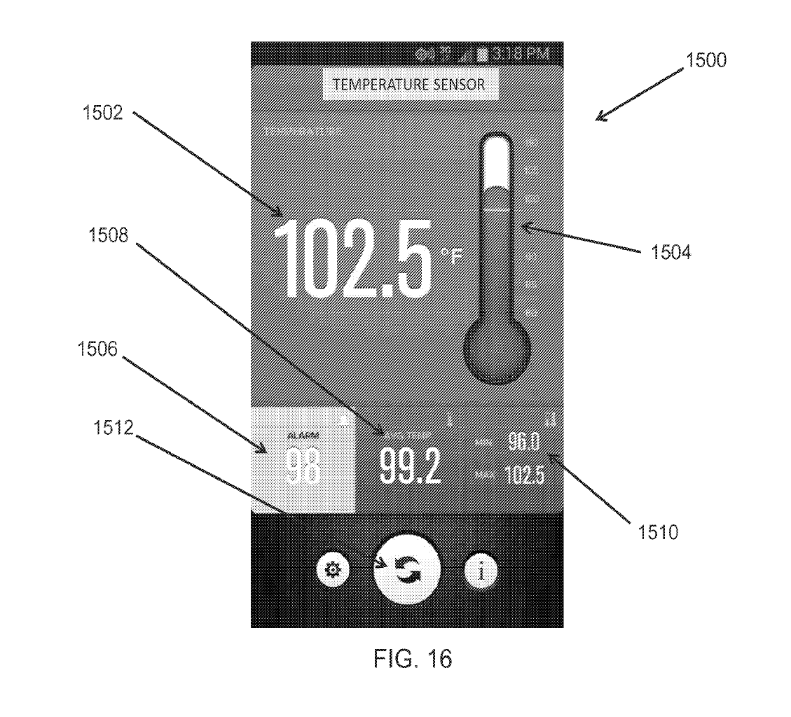

FIG. 16 shows example fields of the homepage 1500. The App can be configured to display a temperature graphic 1504 and thermometer graphic 1504 to indicate to a user the latest temperature measured by the conformal sensor device of the patch. A line on the thermometer graphic 1504 is used to indicate where an alarm is set. The App displays an alarm field 1506 to show the alarm setting (in this example, 98° F.) as a threshold specified by a user or a medical practitioner with consent of the user. If the alarm is set to 98° F. or higher, the alarm can be triggered if the most recent measured conformal sensor data value is above that alarm level. In another example, if the alarm is set to 97° F. or lower, the alarm is triggered if the most recent value falls below that point. In an example, the App can be configured such that the alarm button on the homepage 1506 flashes multiple times or causes the computing device to issue auditory, vibrational and/or other visual alerts when the alarm is triggered. For example, alarm field 1506 may flash 5 times and stay a specified color (such as yellow or red) until the most recent temperature measurement is observed to fall outside the alarm setting range. The App can be used to display an average temperature field 1508 (the average of measured temperatures between the most recent sync and the beginning of the measurement period), and a min/max field 1510 (the high and low temperatures measured over the measurement period). The example temperature App facilitates data transfer from the conformal sensor device in the patch to the computing device using “Sync” button 1512. For example, the computing device can use NFC to receive data collected since the last synchronization, e.g., transferred from an EEPROM memory of the conformal sensor device. The data may be stored to a data base of the computing device. In other examples, the data can be transferred using other technology such as but not limited to Bluetooth® or Wifi.

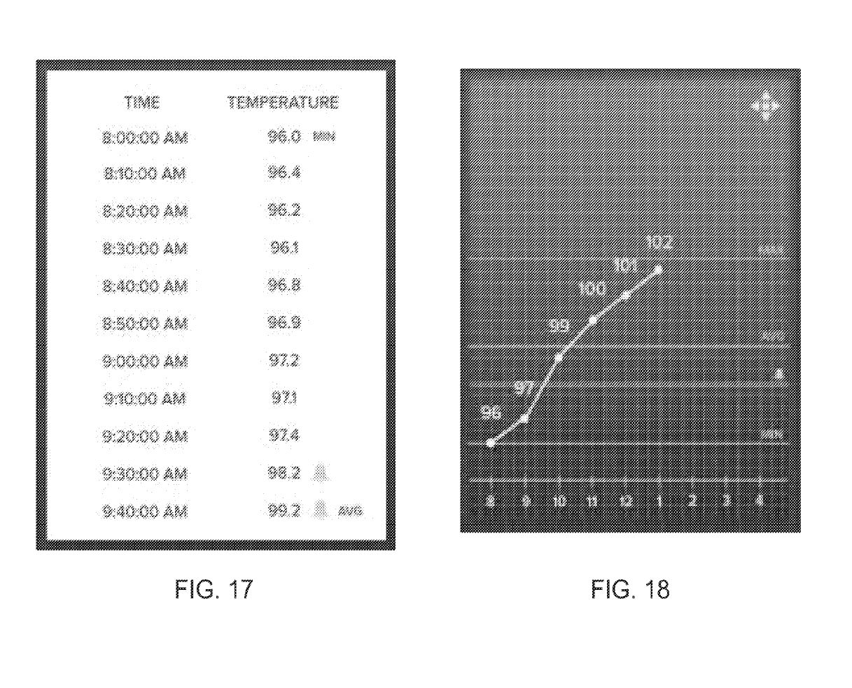

As shown in FIG. 17, the example temperature App can be configured to display a table of any data collected based on measurements of the conformal sensor device, and read from the patch. Alarm indicators can be displayed with the Table based on the user navigating to the table through the Min/Max button or through the Avg button. As shown in FIG. 18, the temperature App also can be configured to show a graphical plot of an averaged value of temperature (representing the points measured within a specified time period). The example plot can include lines to indicate the values for maximum temperature (as specified), average temperature based on the data analysis, and minimum temperature (as specified). The App can be configured to display these different reference lines depending on whether the user navigates to the graph from the Min/Max Button or from the Avg Button.

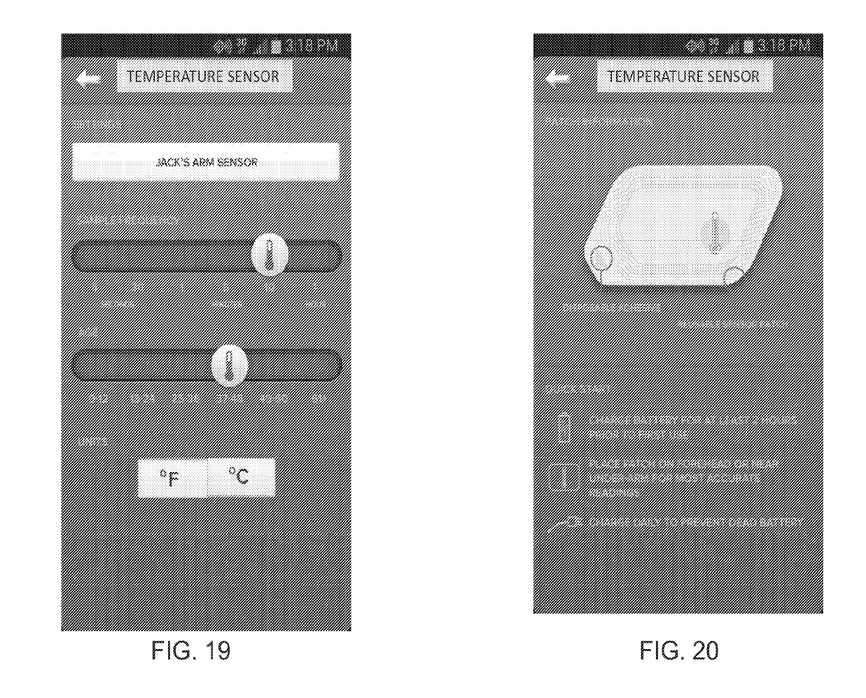

FIG. 19 shows an example settings page that can be used to specify values for the temperature sensor. For example, a data collection frequency or sample measurement frequency can be set using a slider. In an example, altering the slider can directly affect the sampling rate (how often the patch reads skin temperature) on the patch. The sampling frequency can affect the life of the power source of the patch (e.g., the higher the frequency that is set, the longer the battery life for the patch). An example slider is also provided for setting a user's age. The temperature App also allows a user to toggle between temperature scales (i.e., between ° F. and ° C.

FIG. 20 shows an example patch information display that the user can access on the temperature App to provide information about the conformal sensor device and the patch layout. For example, the temperature App can be configured to show a display of the different parts of the patch, how they work, and information that a user can use to place and implement the patch on-body.

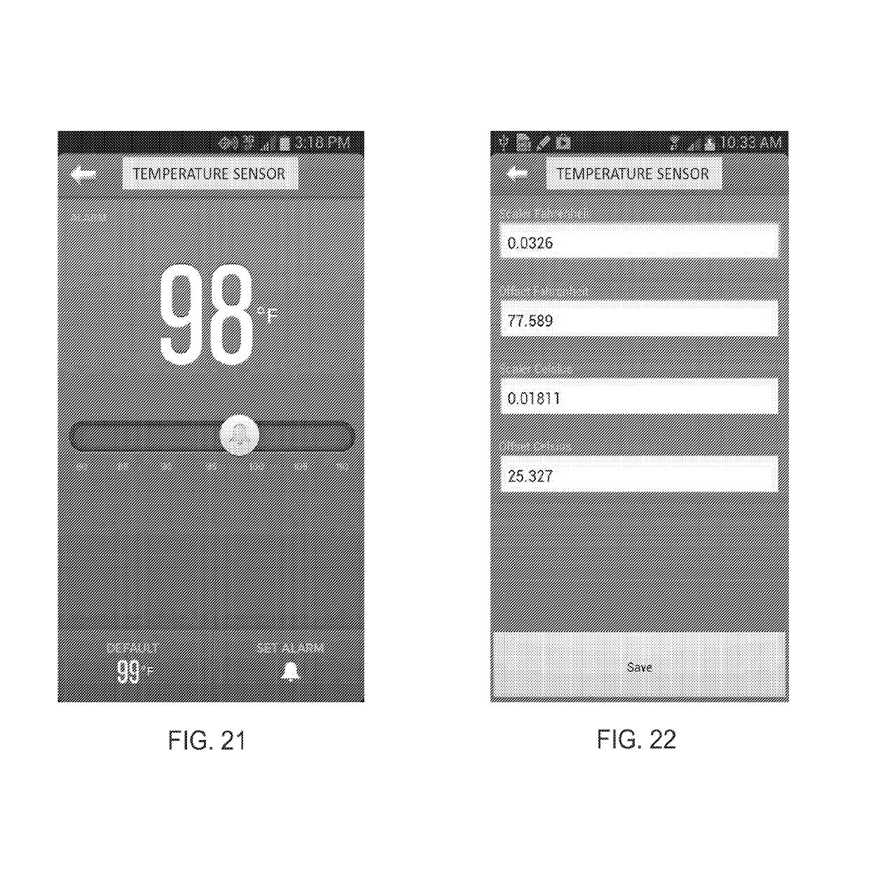

FIG. 21 shows an example alarm display and slider. The example display shows the current alarm set point. A user is allowed to move the slider left or right to change alarm set point. Altering the slider can trigger the alarm if the most recent temp falls above the set point (in this example, for an alarm setting of 98° F. or higher) or if the most recent temp falls below the set point (for alarm sets of 97° F. or lower).

FIG. 22 shows an example of a settings page that can be used to display the values used in the computation of parameters indicating the desired temperature properties based on the measurement data, including values for a scaler Fahrenheit, scaler Celcius, offset Fahrenheit and offset Celcius.

The scaler Fahrenheit value can be a multiplier to the DECIMAL representation of the value on the patch's EEPROM.

° F.=Scaler*hex2dec(Temp Memory Location)+Offset.

Increasing this value can result in higher ° F. values displayed.
The offset Fahrenheit value is added to create a full ° F. temperature that is displayed in-App.

° F.=Scaler*hex2dec(Temp Memory Location)+Offset.

Increasing this value results in higher ° F. values displayed.
The scaler Celcius value can be a multiplier to the DECIMAL representation of the value on the patch's EEPROM.

° C.=Scaler*hex2dec(Temp Memory Location)+Offset.

Increasing this value can result in higher ° F. values displayed.
The offset Celsius value is added to create a full ° C. temperature that is displayed in-App.

° C.=Scaler*hex2dec(Temp Memory Location)+Offset.

Increasing this value results in higher ° C. values displayed.

In a non-limiting example, the analysis engine of the temperature App can be configured to compute the temperature as follows:

° F.=° F. Scaler*Hex2Dec((([7,0]−Samp Time)<<=8)+[15,8])+° F. Offset

*° F. is rounded to the nearest tenth. Default ° F. Scaler=0.0326. Default ° F. Offset=77.589.

° C.=° C. Scaler*Hex2Dec((([7,0]−Samp Time)<<=8)+[15,8])+° C. Offset

*° C. is rounded to the nearest tenth. Default ° C. Scaler=0.01811. Default ° C. Offset=25.327.

Average Temperature:

$$Avg Temp = \frac{\sum All temperature samples}{\# of samples}$$

*Avg Temp resets at the beginning of every day.

Minimum Temperature:

Min Temp=Smallest temperature recorded that day

Maximum Temperature:

Max temp=Largest temperature recorded that day

While this specification contains many specific implementation details, these should not be construed as limitations on the scope of any inventions or of what may be claimed, but rather as descriptions of features specific to particular embodiments of the systems and methods described herein. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

1-24. (canceled)

25. A system for monitoring a surface using a conformal sensor device mounted thereto, the system comprising:
a display device;
a processing unit and associated memory, the processing unit for accessing the memory and executing processor executable instructions stored therein;
a communication module configured to receive data from the conformal sensor device, the data being indicative of an amount of ultraviolet electromagnetic radiation incident on a sensor component of the conformal sensor device; and
an application comprising an analysis engine, the application being executable by the processing unit to initiate the analysis engine to analyze the data to determine one or more parameters, the one or more parameters including a value of computed UV exposure percentage.

26. The system of claim 25, wherein the value of computed exposure percentage parameter is displayed on the display device in the form of a UV exposure wheel.

27. The system of claim 25, wherein the display device is configured to display the value of computed exposure percentage as a numeric number and in the form of a UV exposure wheel.

28. The system of claim 25, wherein the display device is configured to display the one or more parameters determined by the analysis engine thereon.

29. The system of claim 25, wherein the one or more parameters further includes a recommended time remaining for safe UV exposure.

30. The system of claim 29, wherein the recommended time remaining for safe UV exposure is based on a cumulative UVI-minute exposure for a specific day and measured UVA and UVB levels.

31. The system of claim 25, wherein the one or more parameters further include an elapsed time of exposure, a recommended value of SPF, a value for UVA, a value for UVB, or any combination thereof.

32. The system of claim 25, wherein the received data is further indicative of a temperature of a portion of the surface.

33. The system of claim 25, wherein the system is a smartphone.

34. The system of claim 25, further comprising a conformal sensor device configured to transmit data to the communication module.

35. The system of claim 34, wherein the conformal sensor device includes a flexible, stretchable substrate, and wherein the sensor component is disposed on the flexible, stretchable substrate.

36. The system of claim 35, wherein the conformal sensor device further includes at least one stretchable interconnect that electrically couples the sensor component to at least one other component of the conformal sensor device, the at least one other component is one of: a battery, a transmitter, a transceiver, an amplifier, a processing unit, a charger regulator for a battery, a radio-frequency component, a memory, and an analog sensing block.

37. The system of claim 25, wherein the communication module comprises a near-field communication (NFC)-enabled component to receive the data.

38. The system of claim 25, wherein the surface is a portion of a tissue, a fabric, a plant, an artwork, paper, wood, a mechanical tool, or a piece of equipment.

39. A system for monitoring a surface using a conformal sensor device mounted thereto, the system comprising:
a display device;
a processing unit and associated memory, the processing unit for accessing the memory and executing processor executable instructions stored therein;
a communication module configured to receive data from the conformal sensor device, the data being indicative of a temperature of a portion of the surface; and
an application comprising an analysis engine, the application being executable by the processing unit to initiate the analysis engine to analyze the data to determine one or more parameters, the one or more parameters including a latest measured temperature of the portion of the skin.

40. The system of claim 39, wherein the latest measured temperature of the portion of the skin is displayed on the display device in the form of a thermometer graphic.

41. The system of claim 39, wherein the display device is configured to display the latest measured temperature of the portion of the skin as a numeric number and in the form of a thermometer graphic.

42. The system of claim 39, wherein the display device is configured to display the one or more parameters determined by the analysis engine thereon.

43. The system of claim 39, wherein the one or more parameters further includes an average temperature of the portion of the skin, a minimum temperature of the portion of the skin, and a maximum temperature of the portion of the skin.

44. The system of claim 39, wherein the received data is further indicative of an amount of ultraviolet electromagnetic radiation incident on a sensor component of the conformal sensor device.

45. The system of claim 39, wherein the system is a smartphone.

46. The system of claim 39, further comprising a conformal sensor device configured to transmit data to the communication module.

47. The system of claim 46, wherein the conformal sensor device includes a flexible, stretchable substrate, and wherein the sensor component is disposed on the flexible, stretchable substrate.

48. The system of claim 47, wherein the conformal sensor device further includes at least one stretchable interconnect that electrically couples the sensor component to at least one other component of the conformal sensor device, the at least one other component is one of: a battery, a transmitter, a transceiver, an amplifier, a processing unit, a charger regulator for a battery, a radio-frequency component, a memory, and an analog sensing block.

49. The system of claim 39, wherein the communication module comprises a near-field communication (NFC)-enabled component to receive the data.

50. The system of claim 39, wherein the surface is a portion of a tissue, a fabric, a plant, an artwork, paper, wood, a mechanical tool, or a piece of equipment.
